(19) United States
(12) Patent Application Publication
(10) Pub. No.: US 2014/0286945 A1
(43) Pub. Date: Sep. 25, 2014

(54) ENZYME INHIBITOR FOR CANCER TREATMENT

(71) Applicant: The Curators of the University of Missouri, Columbia, MO (US)

(72) Inventors: Carl Freter, Columbia, MO (US); Indira Benakanakere, Columbia, MO (US)

(73) Assignee: The Curators of the University of Missouri, Columbia, MO (US)

(21) Appl. No.: 14/352,950

(22) PCT Filed: Oct. 22, 2012
(86) PCT No.: PCT/US2012/061282

§ 371 Date: Apr. 18, 2014

Related U.S. Application Data
(60) Provisional application No. 61/627,901, filed on Oct. 20, 2011.
(60) Provisional application No. 61/692,444, filed on Aug. 23, 2012.

(51) Int. Cl. A61K 31/454; A61K 31/439; A61K 31/137; A61K 31/138; A61K 31/7076; A61K 39/395

(52) U.S. Cl. CPC A61K 31/454; A61K 31/7076; A61K 39/3955; A61K 31/137; A61K 31/138; A61K 31/439
USPC 424/133.1; 514/326; 514/48; 514/655; 514/651; 514/305

(57) ABSTRACT

Disclosed herein is a new and improved therapy for the treatment of diseases including cancer, which comprises the step of altering cell membrane lipid composition by treating a cancer cell with an enzyme inhibitor which inhibits enzymes regulating the cholesterol biosynthetic pathway. The method may also be employed in combination with existing chemotherapeutic agents to combat the drug resistance and enhance the therapeutic efficacy of conventional therapy.

This application claims the benefit of priority of U.S. provisional applications No. 61/627,901, filed on Oct. 20, 2011, and No. 61/692,444, filed on Aug. 23, 2012, the disclosures of which are hereby incorporated by reference as if written herein in their entireties.

Disclosed herein are pharmaceuticals and treatments for diseases including cancer, more specifically to enzyme inhibitors targeting the enzymes in the mevalonate pathway, to sensitize variety of tumor cells to chemotherapy.

Enzymes in mevalonate pathway. Statins have been used to lower cholesterol in humans by inhibiting the enzyme HMGCoA reductase in the mevalonate pathway. However, inhibition of cholesterol at this stage in the pathway has shown to have a number of side effects as it also interferes with synthesis of prenylated proteins, Heme A, dolichol, ubiquinone, and other byproducts of the mevalonate pathway in addition to cholesterol, because the Mevalonate pathway is shared by other major metabolic pathways. Enzymes, such as squalene synthase, squalene epoxidase, and oxidosqualene cyclase, are involved in later stages of the mevalonate pathway (as shown in FIG. 1), which more selectively regulate cholesterol synthesis.

During the studies to develop antifungal drugs and lipid lowering compounds, many different enzyme inhibitors of the mevalonate pathway have been reported. For example, BIBB 515 and Ro-48-8071 are known oxidosqualene cyclase inhibitors shown to lower lipids in the rat, hamster, squirrel monkey and minipig models due to its inhibition of LDL production; terbinafine, a squalene epoxidase inhibitor, is an active reagent used to treat fungal infections in humans; and YM-53601, a squalene synthase inhibitor, has been used to study lipid lowering in several animal species. These exemplary inhibitors are relatively safe with no reported toxicity in rat studies. However, there have been no reported uses of these enzyme inhibitors in cancer therapy of any kind.

Cancer Treatment and Therapy. There have been many biochemical and mechanical approaches to combat cancer. Some cancer cells have also developed resistance to the existing chemotherapeutic drugs, which renders the chemotherapeutic drugs ineffective or less effective. However, there are no studies on altering cancer cell membrane lipid composition as a direct treatment approach or for enhancing the therapeutic effects of existing drugs.

Therefore, there is a need for a new and improved method for anticancer therapeutics by altering cancer cell membrane lipid composition. There is also a need for providing a new and improved combination therapy for anticancer treatment by altering cancer cell membrane lipid composition in combination with the existing chemotherapeutic treatments to overcome resistance.

Disclosed herein is a new method for anticancer treatment by altering cancer cell membrane lipid composition. The method comprises the step of altering cancer cell membrane lipid composition by treating the cells with inhibitors of enzymes regulating the cholesterol biosynthetic pathway in the later stage of the mevalonate pathway. The method may further comprise the step of treating the cancer cells with an existing chemotherapeutic agent either sequentially or simultaneously with a cholesterol biosynthetic pathway inhibitor. According to one embodiment of the invention, the enzyme may be selected from the group consisting of oxidosqualene cyclase, squalene epoxidase, and squalene synthase, any combination thereof. According to another embodiment of the invention, the inhibitor may be selected from the group consisting of BIBB515, Ro-48-8071, Terbinafine, YM-53601, TAK-475, and any derivative or combination thereof.

### BRIEF DESCRIPTION OF DRAWINGS

FIG. 1 is a flow chart illustration of the Mevalone pathway.

FIG. 2 is a flow-cytometric analysis of CD-20 and CD-54 in Mec-2 cells treated with BIBB515 (BIBB) alone, Fludarabine (FLU, 10 μM) alone, and a combination of both; intensity of stain is on the Y-axis.

FIG. 3 shows Mec-2 cell viabilities after BIBB515 and combination treatments at 24 and 72 hours; percent viable cells is shown on the Y-axis.

FIG. 4 shows the cell viability data after BIBB515 and various combinations of treatments including Fludarabine (FLU, 10 μM) and Rituximab (RIT); cellular proliferation is on the Y-axis.

FIG. 5 shows the cell viability data after treatment of Terbinafine (TB) and in combination with Fludarabine (FLU, 10 μM); cellular proliferation is on the Y-axis.

FIG. 6 shows the cell viability data after treatment of Ro-48-8071 (RO) and in combination with Fludarabine (FLU, 10 μM); cellular proliferation is on the Y-axis.

FIG. 7 shows the cell viability data after treatments of YM-53601 (YM, 100 nM) and in combination with Fludarabine; absorbance×1000 is on the Y-axis.

FIG. 8 shows cell viability data on Wac-3 cells after BIBB515 and in various combinations of treatments including Fludarabine (FLU, 10 μM) and Rituximab.

FIG. 9 shows Raji cell viability after treatment with BIBB515 (BIBB, various concentrations) and BIBB515 in combination with Fludarabine (FLU) at 72 hours; cellular proliferation is on the Y-axis.

FIG. 10 shows Raji cell viability after treatment with Terbinafine and in combination with Fludarabine (FLU) at 72 hours; cellular proliferation is on the Y-axis.

FIG. 11 shows MCF-7 cell proliferation data after treatment with BIBB515 (BIBB) Tamoxifen (TAM), Anastrazole (Aramidex, ARI), and various combinations thereof at several concentrations; cellular proliferation is on the Y-axis.

FIG. 12 shows cell proliferation data of multiple myeloma cell line U226B1 after treatment of Ro-48-8071 (RO) separately and in combination with Fludarabine; cellular proliferation is on the Y-axis.

FIG. 13 shows the Mec-2 cell viability data after TAK-475 and various combinations of treatments including Fludarabine (FLU) and Rituximab (RIT); absorbance at 570 nM is on the Y-axis.

FIG. 14 shows the Wac-3 cell viability data after TAK-475 (TAK) and various combinations of treatments including Fludarabine (FLU, 5 μM) and Rituximab (RIT).

FIG. 15 shows the cell viability of dog lymphoma cells treated with BIBB515 (BIBB, 10 μM) alone and in combination with lomustine (100 nM); absorbance×100 is on the Y-axis.

FIG. 16 shows the cell viability of dog lymphoma cells treated with BIBB515 (BIBB, 10 μM) alone and in combination with Chlorambucil (100 nM); absorbance×100 is on the Y-axis.

FIG. 17 shows the cell viability of patient (n=6) CLL cells treated with a cholesterol inhibitor (BIBB515, YM, TAK-475 and Ro-48-8071) alone and in combination with Fludarabine (FLU) and Rituximab (RIT); cellular viability is on the Y-axis.

FIG. 18 shows the cell viability of patient (n=4) CLL cells treated with TAK-475 (TAK) alone and in combination with Fludarabine (FLU) and Rituximab (RIT); cellular viability is on the Y-axis.

FIG. 19 shows the reduction of lysophosphatidylcholines (LPC) by BIBB515 (BIBB) and YM-53601 (YM) as determined by mass spectrometry; nanomole/3 million cells is on the Y-axis.

FIG. 20 shows the increase of phosphatidylcholines (PC) by BIBB515 (BIBB) and YM-53601 (YM) as determined by mass spectrometry; nanomole/3 million cells is on the Y-axis.

FIG. 21 shows the increase of sphingomyelin/dihydrosphingomyelin by BIBB515 (BIBB) and YM-53601 (YM) as determined by mass spectrometry; nanomole/3 million cells is on the Y-axis.

FIG. 22 shows the increase of ether-linked phosphatidylcholines by BIBB515 (BIBB) and YM-53601 (YM) as determined by mass spectrometry; nanomole/3 million cells is on the Y-axis.

### DETAILED DESCRIPTION OF INVENTION

The methods disclosed herein take advantage of breaking down cancer cell defense mechanisms by altering the membrane lipid profile leading to a direct treatment approach or to enhancing the therapeutic effect of existing drugs. Certain aspects of the invention suggests that the alteration in membrane fluidity and/or permeability is disrupting a variety of cellular processes, such as protein rafts, cell surface receptors, mitotic anchorage points, cleavage furrow, regulation of cell surface markers, and most importantly drug influx through WNT and ENT transporters as well efflux through multidrug resistance-associated protein transporter, i.e., MRP subfamily and P-glycoprotein.

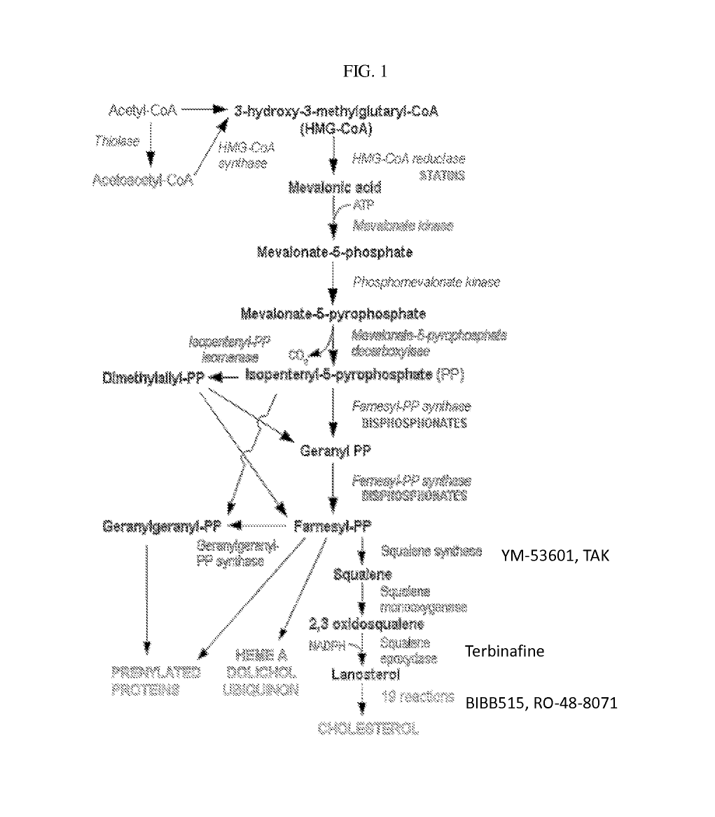

Certain aspects of the invention teach that inhibiting the activities of the enzymes regulating the cholesterol biosynthetic pathway (the terminal stage of the mevalone pathway, as shown in FIG. 1), such as oxidosqualene cyclase, squalene epoxidase, and squalene synthase, has a positive effect on inhibiting the cancer cell growth and viability. Such enzyme inhibitors, including BIBB515, Ro-48-8071, Terbinafine, TAK-475, and YM-5301, have been found to be effective agents alone in reducing the cancer cell proliferation in treatment of certain cancer cell lines. The studies also indicate that the enzyme inhibitors targeting the cholesterol biosynthetic pathway up-regulate CD-20 and CD-54 on the cancer cell membrane; thus, targeting cells with anti-CD-20 and anti-CD-54 after induction by cholesterol biosynthetic pathway inhibitors may be a viable strategy for treatment. The enzyme inhibitors targeting the cholesterol biosynthetic pathway are further studied in combination with existing chemotherapeutic agents, sequentially or simultaneously. The studies find that treating drug-resistant cancer cell lines with the enzyme inhibitors sensitizes the cancer cells, so that the cancer cells respond to the chemotherapeutic agents. Furthermore, the treatments of cancer cell lines, which are not drug-resistant, with the enzyme inhibitors precondition the cancer cells so that the cancer cells respond to the chemotherapeutic agents at lower doses than those of the current practices. Thus, the results demonstrate that inhibiting the cholesterol biosynthetic pathway sensitizes the cancer cells to chemotherapeutic agents and enhance the therapeutic efficacy of the existing drugs.

Accordingly, disclosed herein are new combinations of compounds, new pharmaceutical compositions including combination pharmaceutical compositions, certain of which have been found to inhibit the cholesterol biosynthetic pathway and/or modify the lipid composition of cellular membranes, have been discovered, together with methods of using the compounds including methods for the treatment of diseases such as cancer in a patient by administering the compounds.

Certain aspects and embodiments of the invention are disclosed below. For each method of treatment of disease or effecting of a change in cellular phenotype (e.g., reducing cellular proliferation) using a compound or composition, the corresponding use of that compound or composition in the treatment of disease, as well as the compound or composition for use in the manufacture of a medicament for the treatment of disease, are contemplated.

In one aspect, provided herein is a method of treatment of cancer comprising the administration of a therapeutically effective amount of a cholesterol biosynthetic pathway inhibitor.

In certain embodiments, the cancer is treatment-resistant.

In certain embodiments, the inhibitor inhibits an enzyme regulating the cholesterol biosynthetic pathway.

In certain embodiments, the enzyme is chosen from oxidosqualene cyclase, squalene epoxidase, and squalene synthase, or any combination thereof. In certain embodiments, the inhibitor is an oxidosqualene cyclase inhibitor. In certain embodiments, the inhibitor is a squalene epoxidase inhibitor. In certain embodiments, inhibitor is a squalene synthase inhibitor. In certain embodiments, the inhibitor is chosen from BIBB515, Ro-48-8071, Terbinafine, YM-53601, TAK-475, and any derivative or combination thereof.

In certain embodiments, the cancer is a hematologic cancer. In further embodiments, the hematologic cancer is chosen from leukemia, lymphoma, and myeloma. In certain embodiments, the cancer is lymphoma. In further embodiments, the lymphoma is chosen from Burkitt's lymphoma, cutaneous T-cell lymphoma (CTCL), T-cell lymphoma, Hodgkin's lymphoma, and non-Hodgkin's lymphoma. In certain embodiments, the cancer is leukemia. In further embodiments, the leukemia is chosen from acute myelogenous leukemia, acute monocytic leukemia, acute lymphoblastic leukemia, chronic myelogenous leukemia, and chronic myelogenous leukemia. In certain embodiments, the cancer is myeloma. In further embodiments, the myeloma is chosen from multiple myeloma and plasmacytoma. In certain embodiments, the cancer is treatment-resistant.

In other embodiments, the cancer is non-hematologic. In certain embodiments, the cancer is a solid tumor. In certain embodiments, the solid tumor is breast cancer. In further embodiments, the breast cancer is treatment-resistant.

Also provided herein is a method of treatment of disease comprising the administration of a therapeutically effective amount of an inhibitor of an enzyme chosen from oxidosqualene cyclase, squalene epoxidase, and squalene synthase, or any combination thereof. In certain embodiments, the enzyme chosen from squalene epoxidase, and squalene synthase, or any combination thereof.

In certain embodiments, the disease is cancer. In further embodiments, the cancer is treatment-resistant.

In certain embodiments, the inhibitor is an oxidosqualene cyclase inhibitor. In certain embodiments, the inhibitor is a squalene epoxidase inhibitor. In certain embodiments, the inhibitor is a squalene synthase inhibitor. In certain embodiments, the inhibitor is chosen from BIBB515, Ro-48-8071, Terbinafine, YM-53601, TAK-475, and any derivative or combination thereof

In certain embodiments, the cancer is a hematologic cancer. In further embodiments, the hematologic cancer is chosen from leukemia, lymphoma, and myeloma. In certain embodiments, the cancer is lymphoma. In further embodiments, the lymphoma is chosen from Burkitt's lymphoma, cutaneous T-cell lymphoma (CTCL), T-cell lymphoma, Hodgkin's lymphoma, and non-Hodgkin's lymphoma. In certain embodiments, the cancer is leukemia. In further embodiments, the leukemia is chosen from acute myelogenous leukemia, acute monocytic leukemia, acute lymphoblastic leukemia, chronic myelogenous leukemia, and chronic myelogenous leukemia. In certain embodiments, the cancer is myeloma. In further embodiments, the myeloma is chosen from multiple myeloma and plasmacytoma. In certain embodiments, the cancer is treatment-resistant.

In certain embodiments, the cancer is a solid tumor. In certain embodiments, the solid tumor is breast cancer. In further embodiments, the breast cancer is treatment-resistant.

Also provided herein is a method of reducing cellular proliferation or viability comprising the step of altering membrane lipid composition of a cell. In one aspect, provided herein is a method of reducing cancer cell proliferation or viability comprising the step of altering membrane lipid composition of a cancer cell. In certain embodiments, the cancer cell is treatment-resistant. In certain embodiments, the method further comprises the step of treating the cancer cell with an existing chemotherapeutic agent or therapeutic monoclonal antibody, sequentially or simultaneously with the altering step.

In certain embodiments, the altering is achieved by inhibiting the cholesterol biosynthetic pathway. In certain embodiments, this inhibiting step further comprises the step of inhibiting an enzyme regulating the cholesterol biosynthetic pathway. In certain embodiments, the enzyme is chosen from oxidosqualene cyclase, squalene epoxidase, and squalene synthase, and any combination thereof. In certain embodiments, the inhibitor is an oxidosqualene cyclase inhibitor. In certain embodiments, the inhibitor is a squalene epoxidase inhibitor. In certain embodiments, inhibitor is a squalene synthase inhibitor. In certain embodiments, the inhibitor is chosen from BIBB515, Ro-48-8071, Terbinafine, YM-53601, TAK-475, and any derivative or combination thereof.

Also provided herein is a method of treatment of cancer comprising the administration of:
an inhibitor of an enzyme regulating membrane cholesterol or membrane lipid composition; and
an additional, chemotherapeutic agent, either together or sequentially.

In certain embodiments, the inhibitor is a cholesterol biosynthetic pathway inhibitor. In further embodiments, the inhibitor targets an enzyme chosen from oxidosqualene cyclase, squalene epoxidase, and squalene synthase, or any combination thereof. In certain embodiments, the inhibitor is an oxidosqualene cyclase inhibitor. In certain embodiments, the inhibitor is a squalene epoxidase inhibitor. In certain embodiments, the inhibitor is a squalene synthase inhibitor. In certain embodiments, the inhibitor is chosen from BIBB515, Ro-48-8071, Terbinafine, YM-53601, TAK-475, and any derivative or combination thereof.

In certain embodiments, the additional, chemotherapeutic agent is an agent for the treatment of hematologic malignancies.

In certain embodiments, the additional, chemotherapeutic agent is an inhibitor of DNA synthesis. In certain embodiments, the additional, chemotherapeutic agent is a purine analog. In further embodiments, the additional, chemotherapeutic agent is fludarabine.

In certain embodiments, the additional, chemotherapeutic agent is a CD20-targeting monoclonal antibody. In further embodiments, the additional, chemotherapeutic agent is chosen from ocrelizumab, ofatumumab, and rituximab. In yet further embodiments, the agent is rituximab. In other embodiments, the additional, chemotherapeutic agent is a CD52-targeting monoclonal antibody. In further embodiments, the additional, chemotherapeutic agent is alemtuzumab.

In certain embodiments, the additional, chemotherapeutic agent is an alkylating antineoplastic agent. In further embodiments, the additional, chemotherapeutic agent is chosen from fludarabine and chlorambucil.

In certain embodiments, the additional, chemotherapeutic agent is an aromatase inhibitor. In further embodiments, the additional, chemotherapeutic agent is anastrozole.

In certain embodiments, the additional, chemotherapeutic agent is an estrogen receptor (ER) antagonist or selective ER modulator (SERM). In further embodiments, the additional, chemotherapeutic agent is tamoxifen.

Also provided herein is a method of reducing drug resistance in a cancer cell, and a method of overcoming drug resistance in a cancer cell, comprising the administration of a therapeutically effective amount of an inhibitor of an enzyme regulating membrane cholesterol or membrane lipid composition.

In certain embodiments, the inhibitor inhibits an enzyme regulating the cholesterol biosynthetic pathway. In certain embodiments, the enzyme is chosen from oxidosqualene cyclase, squalene epoxidase, and squalene synthase, or any combination thereof. In certain embodiments, the inhibitor is an oxidosqualene cyclase inhibitor. In certain embodiments, the inhibitor is a squalene epoxidase inhibitor. In certain embodiments, inhibitor is a squalene synthase inhibitor. In certain embodiments, the inhibitor is chosen from BIBB515, Ro-48-8071, Terbinafine, YM-53601, TAK-475, and any derivative or combination thereof.

In certain embodiments, the cancer is a hematologic cancer. In further embodiments, the hematologic cancer is chosen from leukemia, lymphoma, and myeloma. In certain embodiments, the cancer is lymphoma. In further embodiments, the lymphoma is chosen from Burkitt's lymphoma, cutaneous T-cell lymphoma (CTCL), T-cell lymphoma, Hodgkin's lymphoma, and non-Hodgkin's lymphoma. In certain embodiments, the cancer is leukemia. In further embodiments, the leukemia is chosen from acute myelogenous leukemia, acute monocytic leukemia, acute lymphoblastic leukemia, chronic myelogenous leukemia, and chronic myelogenous leukemia. In certain embodiments, the cancer is myeloma. In further embodiments, the myeloma is chosen from multiple myeloma and plasmacytoma. In certain embodiments, the cancer is treatment-resistant.

In other embodiments, the cancer is non-hematologic. In certain embodiments, the cancer is a solid tumor. In certain embodiments, the solid tumor is breast cancer. In further embodiments, the breast cancer is treatment-resistant.

Also provided herein is a method for achieving an effect in a cancer cell comprising the administration of a therapeutically effective amount of a cholesterol biosynthetic pathway inhibitor, wherein the effect is chosen from upregulation of CD20, upregulation of CD54, reduction of a lysophosphatidylcholine, reduction of a diacyl phosphatidylcholine, increase of phosphatidylcholine (PC), increase of a glycosphingolipid, increase of a dihydroglycosphingolipid, increase of an ether-linked phosphatidylcholine, and increase in lipid raft density. In certain embodiments, the effect is upregulation of CD20. In certain embodiments, the effect is upregulation of CD54. In certain embodiments, the effect is reduction of a lysophosphatidylcholine. In certain embodiments, the effect is reduction of a diacyl phosphatidylcholine. In certain embodiments, the effect is increase of phosphatidylcholine. In certain embodiments, the effect is increase of a glycosphingolipid. In certain embodiments, the effect is increase of a dihydroglycosphingolipid. In certain embodiments, the effect is increase of an ether-linked phosphatidylcholine. In certain embodiments, the effect is an increase in lipid raft density.

In certain embodiments, the inhibitor inhibits an enzyme regulating the cholesterol biosynthetic pathway. In certain embodiments, the enzyme is chosen from oxidosqualene cyclase, squalene epoxidase, and squalene synthase, and any combination thereof. In certain embodiments, the inhibitor is an oxidosqualene cyclase inhibitor. In certain embodiments, the inhibitor is a squalene epoxidase inhibitor. In certain embodiments, inhibitor is a squalene synthase inhibitor. In certain embodiments, the inhibitor is chosen from BIBB515, Ro-48-8071, Terbinafine, YM-53601, TAK-475, and any derivative or combination thereof.

In certain embodiments, the cancer is a hematologic cancer. In further embodiments, the hematologic cancer is chosen from leukemia, lymphoma, and myeloma. In certain embodiments, the cancer is lymphoma. In further embodiments, the lymphoma is chosen from Burkitt's lymphoma, cutaneous T-cell lymphoma (CTCL), T-cell lymphoma, Hodgkin's lymphoma, and non-Hodgkin's lymphoma. In certain embodiments, the cancer is leukemia. In further embodiments, the leukemia is chosen from acute myelogenous leukemia, acute monocytic leukemia, acute lymphoblastic leukemia, chronic myelogenous leukemia, and chronic myelogenous leukemia. In certain embodiments, the cancer is myeloma. In further embodiments, the myeloma is chosen from multiple myeloma and plasmacytoma. In certain embodiments, the cancer is treatment-resistant.

In other embodiments, the cancer is non-hematologic. In certain embodiments, the cancer is a solid tumor. In certain embodiments, the solid tumor is breast cancer. In further embodiments, the breast cancer is treatment-resistant.

Also provided herein is a pharmaceutical composition comprising
an inhibitor of an enzyme regulating membrane cholesterol or membrane lipid composition; and
an additional, chemotherapeutic agent,
   together with a pharmaceutically acceptable carrier.

In certain embodiments, the inhibitor is a cholesterol biosynthetic pathway inhibitor.

In further embodiments, the inhibitor targets an enzyme chosen from oxidosqualene cyclase, squalene epoxidase, and squalene synthase, or any combination thereof. In certain embodiments, the inhibitor is an oxidosqualene cyclase inhibitor. In certain embodiments, the inhibitor is a squalene epoxidase inhibitor. In certain embodiments, the inhibitor is a squalene synthase inhibitor. In certain embodiments, the inhibitor is chosen from BIBB515, Ro-48-8071, Terbinafine, YM-53601, TAK-475, and any derivative or combination thereof.

In certain embodiments, the additional, chemotherapeutic agent is an agent for the treatment of hematologic cancer.

In certain embodiments, the hematologic cancer is chosen from leukemia, lymphoma, and myeloma. In certain embodiments, the cancer is lymphoma. In further embodiments, the lymphoma is chosen from Burkitt's lymphoma, cutaneous T-cell lymphoma (CTCL), T-cell lymphoma, Hodgkin's lymphoma, and non-Hodgkin's lymphoma. In certain embodiments, the cancer is leukemia. In further embodiments, the leukemia is chosen from acute myelogenous leukemia, acute monocytic leukemia, acute lymphoblastic leukemia, chronic myelogenous leukemia, and chronic myelogenous leukemia. In certain embodiments, the cancer is myeloma. In further embodiments, the myeloma is chosen from multiple myeloma and plasmacytoma. In certain embodiments, the cancer is treatment-resistant.

In certain embodiments, the additional, chemotherapeutic agent is an agent for the treatment of a solid tumor. In certain embodiments, the solid tumor is breast cancer. In further embodiments, the breast cancer is treatment-resistant.

In certain embodiments, the additional, chemotherapeutic agent is an inhibitor of DNA synthesis. In certain embodiments, the additional, chemotherapeutic agent is a purine analog. In further embodiments, the additional, chemotherapeutic agent is chosen from fludarabine.

In certain embodiments, the additional, chemotherapeutic agent is a CD20-targeting monoclonal antibody. In further embodiments, the additional, chemotherapeutic agent is chosen from ocrelizumab, ofatumumab, and rituximab. In yet further embodiments, the agent is rituximab. In other embodiments, the additional, chemotherapeutic agent is a CD52-targeting monoclonal antibody. In further embodiments, the additional, chemotherapeutic agent is alemtuzumab.

In certain embodiments, the additional, chemotherapeutic agent is an alkylating antineoplastic agent. In further embodiments, the additional, chemotherapeutic agent is chosen from fludarabine and chlorambucil.

In certain embodiments, the additional, chemotherapeutic agent is an aromatase inhibitor. In further embodiments, the additional, chemotherapeutic agent is anastrozole.

In certain embodiments, the additional, chemotherapeutic agent is an estrogen receptor antagonist or selective ER modulator (SERM). In further embodiments, the additional, chemotherapeutic agent is tamoxifen.

In certain embodiments, provided herein are methods of treatment of diseases, for example proliferative diseases including cancers, comprising the administration in combination of an agent which inhibits an enzyme regulating membrane cholesterol or membrane lipid composition, such as a cholesterol biosynthetic pathway inhibitor, and an additional agent which is chemotherapeutic. The two agents may be given together or sequentially, and separately or as part of a single formulation, where appropriate. Examples include:

| | |
|---|---|
| Example | Agents for Use in Combination |

| | | | |
|---|---|---|---|
| 1 | BIBB515 | Fludarabine | |
| 2 | BIBB515 | Fludarabine | Rituximab |
| 3 | BIBB515 | Rituximab | |
| 4 | BIBB515 | Tamoxifen | |
| 5 | BIBB515 | Anastrozole | |
| 6 | BIBB515 | Lomustine | |
| 7 | BIBB515 | Chlorambucil | |
| 8 | Terbinafine | Fludarabine | |
| 9 | Terbinafine | Fludarabine | Rituximab |
| 10 | Terbinafine | Rituximab | |
| 11 | Terbinafine | Tamoxifen | |
| 12 | Terbinafine | Anastrozole | |
| 13 | Terbinafine | Lomustine | |
| 14 | Terbinafine | Chlorambucil | |
| 15 | TAK-475 | Fludarabine | |
| 16 | TAK-476 | Fludarabine | Rituximab |
| 17 | TAK-477 | Rituximab | |
| 18 | TAK-478 | Tamoxifen | |
| 19 | TAK-479 | Anastrozole | |
| 20 | TAK-480 | Lomustine | |
| 21 | TAK-481 | Chlorambucil | |
| 22 | Ro-48-8071 | Fludarabine | |
| 23 | Ro-48-8071 | Fludarabine | Rituximab |
| 24 | Ro-48-8071 | Rituximab | |
| 25 | Ro-48-8071 | Tamoxifen | |
| 26 | Ro-48-8071 | Anastrozole | |
| 27 | Ro-48-8071 | Lomustine | |
| 28 | Ro-48-8071 | Chlorambucil | |
| 29 | YM-53601 | Fludarabine | |
| 30 | YM-53601 | Fludarabine | Rituximab |
| 31 | YM-53601 | Rituximab | |
| 32 | YM-53601 | Tamoxifen | |
| 33 | YM-53601 | Anastrozole | |
| 34 | YM-53601 | Lomustine | |
| 35 | YM-53601 | Chlorambucil | |

These examples are meant to illustrate the concept that altering membrane lipid content by inhibiting certain enzymes in the cholesterol biosynthetic pathway can not only treat disease, but also sensitize certain diseased cells such as cancer cells to chemotherapeutics, even when they are or have become resistant to treatment. As the studies herein demonstrate, the benefit of co-administration of cholesterol biosynthetic pathway inhibitors and chemotherapeutics can be additive and even synergistic. Additional cholesterol biosynthetic pathway inhibitors and chemotherapeutics will be known or identifiable to those of skill in the art, and are expected to have efficacy in the treatment of disease.

In certain embodiments, the additional, chemotherapeutic agent is an inhibitor of DNA synthesis. In further embodiments, it is a purine analog. In yet further embodiments, the agent is fludarabine.

In certain embodiments, the additional, chemotherapeutic agent is a CD20-targeting monoclonal antibody. In further embodiments, the agent is chosen from ocrelizumab, ofatumumab, and rituximab. In yet further embodiments, the agent is rituximab. In other embodiments, the additional, chemotherapeutic agent is a CD52-targeting monoclonal antibody. In further embodiments, the additional, chemotherapeutic agent is alemtuzumab.

In certain embodiments, the additional, chemotherapeutic agent is an aromatase inhibitor such as anastrozole (Arimidex), letrozole (Femara), exemestane (Aromasin), vorozole (Rivizor), formestane (Lentaron), and fadrozole (Afema).

In certain embodiments, the additional, chemotherapeutic agent is an estrogen receptor (ER) antagonist or selective ER modulator (SERM) such as tamoxifen, lasofoxifene, toremifene, and raloxifene. In further embodiments, the agent is tamoxifen.

In certain embodiments, the additional, chemotherapeutic agent is an alkylating antineoplastic agent such as chlorambucil, cyclophosphamide, mechlorethamine, uramustine, melphalan, ifosfamide, nitrosoureas, carmustine, lomustine, streptozocin, alkyl sulfonates, busulfan, thiotepa and its analogues, cisplatin, carboplatin, nedaplatin, oxaliplatin, satraplatin, triplatin tetranitrate, procarbazine, altretamine, and the tetrazines (dacarbazine, mitozolomide, temozolomide). In further embodiments, the agent is chlorambucil.

Also disclosed herein is the use of a compound or composition as disclosed herein in the treatment of a disease as disclosed herein.

Also disclosed herein is a compound or pharmaceutical composition as disclosed herein for use in the manufacture of a medicament for the treatment of a disease as disclosed herein.

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “oxidosqualene cyclase” or OSC is meant to be synonymous with “lanosterol synthase,” i.e., the enzyme that catalyzes the cyclization of 2,3-oxidosqualene to lanosterol.

The term “squalene epoxidase” is meant to be synonymous with “squalene monooxygenase,” i.e., the enzyme that catalyzes the first oxygenation step in sterol biosynthesis, using NADPH and molecular oxygen to oxidize squalene to 2,3-oxidosqualene.

The term “squalene synthase” or SQS is meant to be synonymous with “farnesyl-diphosphate farnesyltransferase (FDFT1),” i.e., the enzyme that catalyzes the first dedicated step of sterol synthesis by converting two units of farnesyl pyrophosphate into squalene.

As used herein, “TAK-475” is synonymous with lapaquistat, which is commercially available and has the following structure:

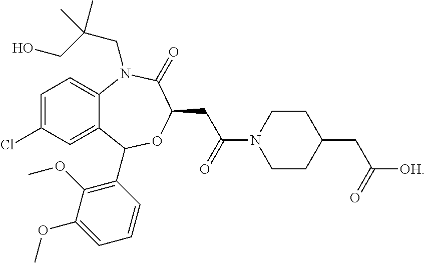 [SMILES: COc1cccc(C2O[C@H](CC(=O)N3CCC(CC(=O)O)CC3)C(=O)N(CC(C)(C)CO)c3ccc(Cl)cc32)c1OC]

As used herein, “YM-53601” is commercially available and has the following structure:

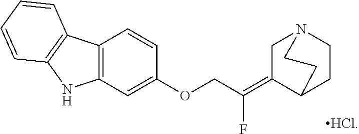 [SMILES: F/C(COc1ccc2c(c1)[nH]c1ccccc12)=C1/CN2CCC1CC2]

As used herein, “Ro-48-8071” is commercially available and has the following structure:

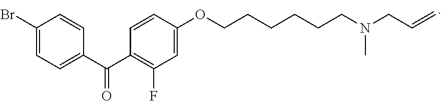 [SMILES: C=CCN(C)CCCCCCOc1ccc(C(=O)c2ccc(Br)cc2)c(F)c1]

As used herein, “terbinafine” (brand names include Lamisil and Terbinex) is commercially available and has the following structure:

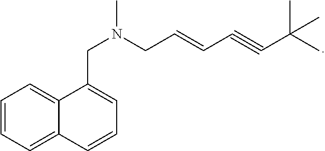 [SMILES: CN(C/C=C/C#CC(C)(C)C)Cc1cccc2ccccc12]

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “treatment-resistant cancer”—in most embodiments, “drug resistant cancer”—refers to cancers which are both intrinsically treatment-resistant, such as, for example, triple negative breast cancer, and cancers which have acquired treatment resistance after one or more courses of chemotherapy. A person skilled in the art (e.g., a clinician or oncologist) will recognize when a cancer is or has become treatment resistant. For example, cancer may be considered treatment resistant when cancer progression continues despite administration of a drug which either a) was previously effective in that patient, or b) is effective in the average patient population of the cancer.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid.

A salt of a compound can be made by reaction of the appropriate compound, in the form of the free base, with the appropriate acid, or by the reacting of the free acid with an appropriate base.

While it may be possible for the compounds of the subject invention to be administered as the raw chemical, it is also possible to present them as pharmaceutical formulations (equivalently, “pharmaceutical compositions”). Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, intraadiposal, intraarterial, intracranial, intralesional, intranasal, intraocular, intrapericardial, intraperitoneal, intrapleural, intraprostatical, intrarectal, intrathecal, intratracheal, intratumoral, intraumbilical, intravaginal, intravesicular, intravitreal, and intramedullary), intraperitoneal, rectal, topical (including, without limitation, dermal, buccal, sublingual, vaginal, rectal, nasal, otic, and ocular), local, mucosal, sublingual, subcutaneous, transmucosal, transdermal, transbuccal, transdermal, and vaginal; liposomal, in cremes, in lipid compositions, via a catheter, via a lavage, via continuous infusion, via infusion, via inhalation, via injection, via local delivery, via localized perfusion, bathing target cells directly, or any combination thereof. Administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as hard or soft capsules, wafers, cachets or tablets each containing a predetermined amount of the active ingredient; as a powder or granules; as a syrup, elixir, solution, or a suspension in an aqueous liquid or a non-aqueous liquid; or as an oil-in-water liquid emulsion, a water-in-oil liquid emulsion, or a compound dispersed in a liposome. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide delayed, slowed, or controlled release or absorption of the active ingredient therein. Compositions may further comprise an agent that enhances solubility or dispersability. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Depending on the route of administration, the compounds, or granules or particles thereof, may be coated in a material to protect the compounds from the action of acids and other natural conditions that may inactivate the compounds.

The compounds may be formulated for parenteral administration by injection, e.g., by bolus injection or continuous infusion, either to the body or to the site of a disease or wound. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. To administer the therapeutic compound by other than parenteral administration, it may be necessary to coat the compound with, or co-administer the compound with a material to prevent its inactivation (for example, via liposomal formulation).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations of the present invention may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenan, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected on the basis of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

In certain topical embodiments, formulations are prepared using a buffering system that maintains the formulation at a pH of about 4.5 to a pH of about 8. In further embodiments, the pH is from 7 to 8.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof), and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The therapeutic compound may also be administered intraspinally or intracerebrally. Dispersions for these types of administrations can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and required other ingredients to be pharmacologically sound. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

It should be understood that in addition to the ingredients particularly mentioned above, the formulations described above may include other agents conventional in the art having regard to the type of formulation in question, for example those suitable for oral administration may include flavoring agents.

Compounds may be administered at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

Preferred unit dosage formulations are those containing an effective dose, as herein below recited, or an appropriate fraction thereof, of the active ingredient. In certain embodiments, a formulation of the present invention is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

The amount of active ingredient that may be combined with the carrier materials to produce a single dosage form will vary depending upon the host treated and the particular mode of administration. Similarly, the precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for breast cancer involving administration of one of the compounds described herein, increased therapeutic benefit may result by also providing the patient with another therapeutic agent for breast cancer. In any case, regardless of the disease, disorder or condition being treated, the overall benefit experienced by the patient may simply be additive of the two therapeutic agents or the patient may experience a synergistic benefit.

Effective combination therapy may be achieved with a single composition or pharmacological formulation that includes both agents, or with two distinct compositions or formulations, at the same time, wherein one composition includes a compound of the present disclosure, and the other includes the second agent(s). Alternatively, the therapy may precede or follow the other agent treatment by intervals ranging from minutes to months. Administration of the compounds of the present disclosure to a patient will follow general protocols for the administration of pharmaceuticals, taking into account the toxicity, if any, of the drug. It is expected that the treatment cycles would be repeated as necessary.

Specific, non-limiting examples of possible combination therapies include use of certain compounds disclosed herein with one or more agents chosen from: aromatase inhibitors, antiestrogens, anti-progestins, anti-androgens, or gonadorelin agonists, topoisomerase land 2 inhibitors, microtubule active agents, alkylating agents, antineoplastic, antimetabolite, dacarbazine (DTIC), or platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors.

For the treatment of oncologic diseases and solid tumors, compounds disclosed herein may be administered with, for example, an agent chosen from: dacarbazine (DTIC), alkylating agents (e.g., melphalan) anthracyclines (e.g. doxorubicin), corticosteroids (e.g. dexamethasone), Akt inhibitor (e.g. Perifosine), aromatase inhibitors, antiestrogen, anti-androgen, or a gonadorelin agonists, topoisomerase 1 and 2 inhibitors, microtubule active agents, alkylating agents (e.g. cyclophosphamide, temozolomide), antineoplastic antimetabolite, or platinum containing compounds, MITC, nitrosoureas, taxanes, lipid or protein kinase targeting agents, protein or lipid phosphatase targeting agents, anti-angiogenic agents, IMiDs (e.g. thalidomide, lenalidomide), protease inhibitors (e.g. bortezomib, NPI0052), IGF-1 inhibitors, CD40 antibody, Smac mimetics (e.g. telomestatin), FGF3 modulator (e.g. CHIR258), mTOR inhibitor (Rad 001), HDAC inhibitors (e.g. SAHA, Tubacin), IKK inhibitors, P38MAPK inhibitors, HSP90 inhibitor (e.g. 17-AAG), and other multikinase inhibitors (e.g. sorafenib).

Thus, in another aspect, the present invention provides methods for treating diseases or disorders in a human or animal subject in need of such treatment comprising administering to said subject an amount of a compound of the present invention effective to reduce or prevent said disorder in the subject in combination with at least one additional agent for the treatment of said disorder that is known in the art.

Used either as a monotherapy or in combination with other agents, the compounds disclosed herein are useful in the prevention and/or treatment of cancers, including hematologic and nonhematologic malignancies including solid tumors, leukemias, lymphomas, and myelomas. These include cancers of the breast, prostate, lung, colon, ovary, pancreas, liver, thyroid, stomach, mouth, throat, tongue, uterus, brain (including, e.g., neuroblastoma and gliobalstoma), skin, kidney, and bladder, as well as blood, lymph nodes, and bone marrow. Lymphomas include Burkitt's lymphoma, cutaneous T-cell lymphoma (CTCL), T-cell lymphoma, Hodgkin's lymphoma, and non-Hodgkin's lymphoma. Leukemias include acute myelogenous leukemia, acute monocytic leukemia, acute lymphoblastic leukemia, chronic myelogenous leukemia, and chronic lymphocytic leukemia.

The cancer may be hormone-dependent or hormone-resistant, such as in the case of breast cancers. In certain embodiments, the cancer is a solid tumor. In certain embodiments, the cancer is and a drug resistant phenotype of a cancer disclosed herein or known in the art.

Additional diseases for which the instant compounds, combinations, methods, and pharmaceutical compositions are useful include autoimmune disorders such as multiple sclerosis, systemic lupus erythematosus, autoimmune hemolytic anemias, and Wegener's granulomatosis, as well as non-oncologic proliferative diseases, such as psoriasis, myloproliferative disorders including essential thrombocytomia ET and polycythemia vera (PVR).

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### Biological Assays

I. Treatment with inhibitors targeting the cholesterol biosynthetic pathway alone and in combination with existing chemotherapeutic agents on Mec-2 cells.

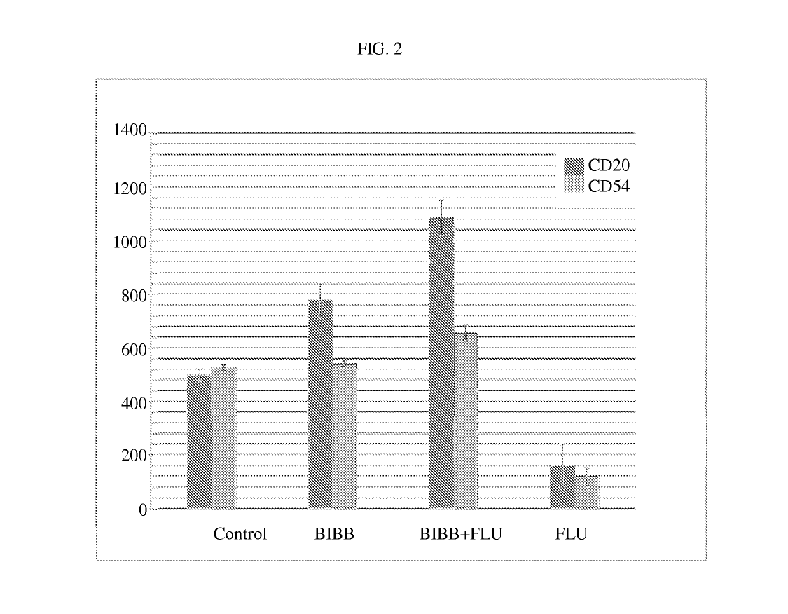

1. Up-regulation of CD-20 and CD54 by treatment of BIBB515 with and without Fludarabine on Mec-2 cells. Refer to FIG. 2, which is a flow-cytometric analysis of CD-20 and CD-54 in Mec-2 cells with the treatments of BIBB515 (oxidosqualene cyclase inhibitor) alone, Fludarabine (an existing chemotherapeutic agent) alone, and in combination with both. Mec-2 cells are B-cell chronic lymphocytic leukemia cells (CLL) and resistant to Fludarabine treatment, which is shown in FIG. 2, whereas comparing to control, Fludarabine treatment down regulates both CD-20 and CD-54. However, as shown in FIG. 2, not only the treatment with BIBB515 alone induces increased CD-20 cell surface expression comparing to Control, but also the combination treatment with BIBB515 and Fludarabine induces about twofold increase in CD-20 expression. Furthermore, while CD-54 cell surface expression maintained at the similar level with BIBB515 alone comparing to Control, CD-54 is moderately up-regulated in the combination treatments. Both CD-20 and CD-54 are cell surface markers on lymphocytes, which were able to produce CDCT (cellular dependent cytotoxicity) when engaged. The genetically engineered humanized antibodies to CD-20 and CD-54 are used currently in clinics for CLL and other malignancies. The results suggest that feasibility of inhibiting cancer cell growth with BIBB515 alone and in combination with agents that target CD-20, CD-52 and/or CD-54.

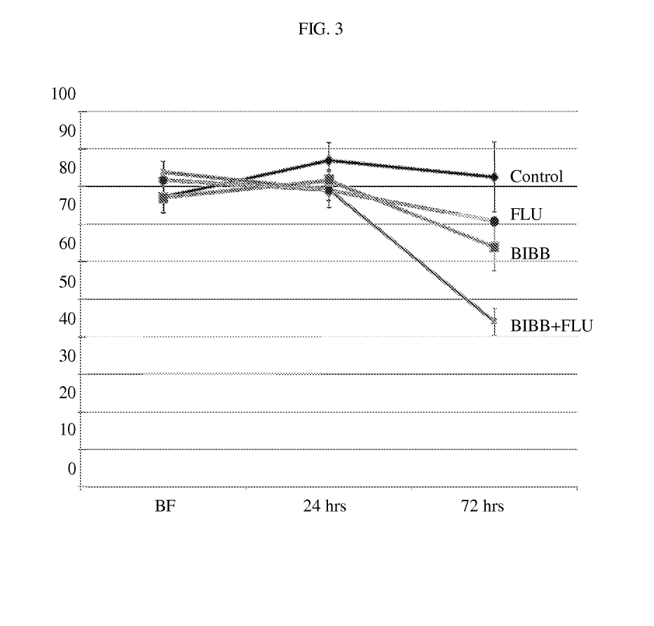

2. Inhibition of cell viability after treatment with BIBB515 alone or in combination with Fludarabine. Refer to FIG. 3, which is a graph showing Mec-2 cell viabilities after BIBB515 treatment and combination treatment at 24 and 72 hours. As shown in FIG. 3, BIBB515 at 10 μM (10⁻⁵) reduces the cell viability from 82% to 65% after 72 hours, while Fludarabine at 10 μM alone does not reduce the cell viability (82%) significantly. However, the combination treatment with BIBB515 and Fludarabine both at 10 μM decreases the cell viability significantly to 37%.

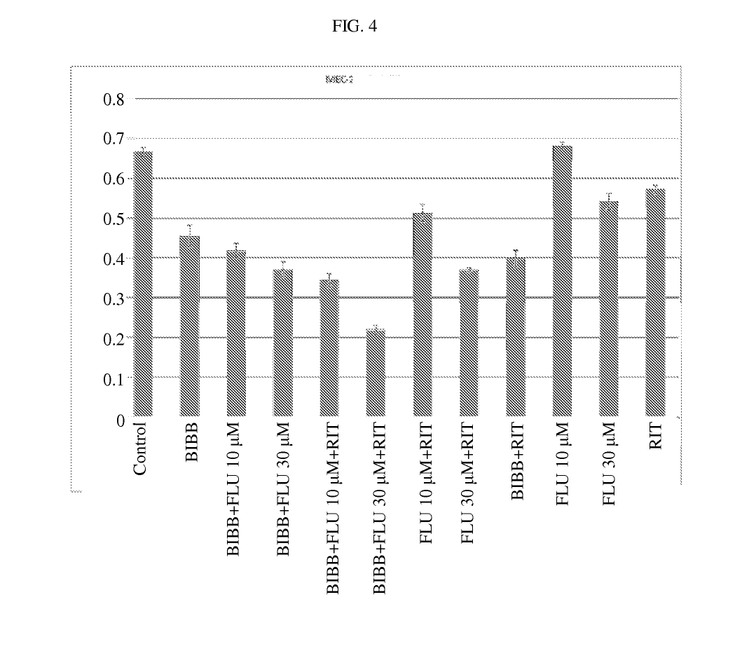

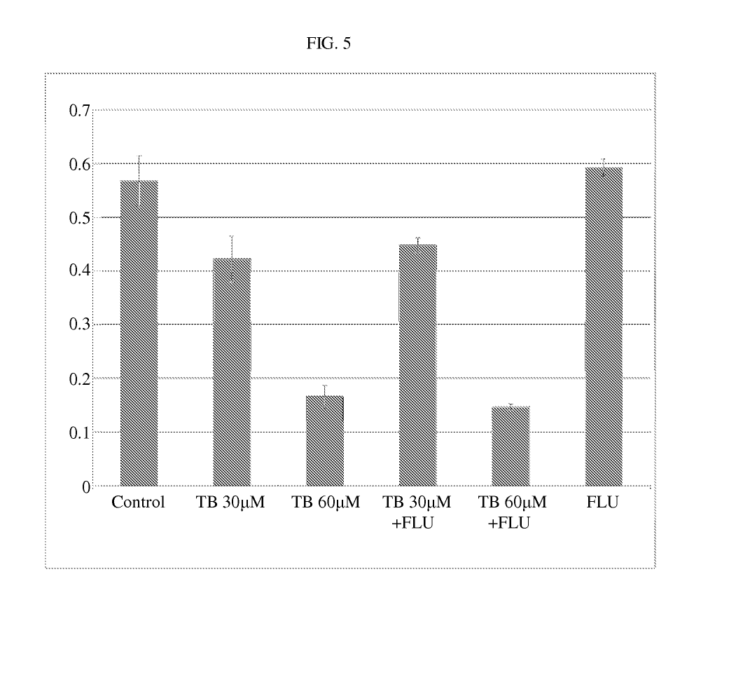

3. Inhibition of cell viability after treatment with BIBB515 alone and in combination with Fludarabine and Rituximab. Refer to FIG. 4, which shows cell viability data with MTT assay after BIBB515 and combination treatments with Fludarabine and Rituximab separately and in combination. As shown in FIG. 4, the viable cell numbers were determined by MTT assay. MTT (3-(4,5-dimethylthiazol-2-y1)-2,5-diphenyl tetrazolium bromide), a water soluble compound, is added to the cell media, and viable cells convert MTT to insoluble formazan, which is then solubilized and the concentration determined by optical density at 570 nM. During the test, Mec-2 cells are treated with BIBB515, Fludarabine, Rituximab (an antiCD-20 monoclonal antibody that is in use in clinics), and in various combinations at different concentrations, and the cell proliferation results are summarized after 72 hours treatments. As seen from the data, BIBB515 at the concentration of 10 μm (10⁻⁵) is able to inhibit cell proliferation, while Mec-2 cells are resistant to Fludarabine and Rituximab when treated alone. Significant cell reductions in cell proliferation are achieved when BIBB515 is used in combination with Fludarabine alone and also in Fludarabine and Rituximab groups. As shown in FIG. 4, cell proliferation by MTT assay is 0.66±0.010 in control compared to 0.37±0.01 in BIBB515+fludarbine and 0.21±0.01in BIBB515+fludarabine+Ritaximab, which indicates a 68% down-regulation of cell proliferation using the later combination treatment.

4. Inhibition on cell viability after treatments with Terbinafine alone and in combination with Fludarabine. Refer to FIG. 5, which shows the cell viability data with MTT assay after treatments with Terbinafine and in combination with Fludarabine. As shown in FIG. 5, the viability of Mec-2 cells is inhibited by Terbinafine (an inhibitor targeting squalene epoxidase in the cholesterol synthetic pathway) significantly at the concentration of 60 μM, whereas the cell proliferation drops from 0.5 in controls to 0.16. The combination treatment with Fludarabine reduces the cell proliferation further, while the cells are not response to the Fludarabine treatment alone. Thus, the results demonstrate that the treatment with Terbinafine, the inhibitor of squalene epoxidase, preconditioned the cancer cells and made it responsive to Fludarabine.

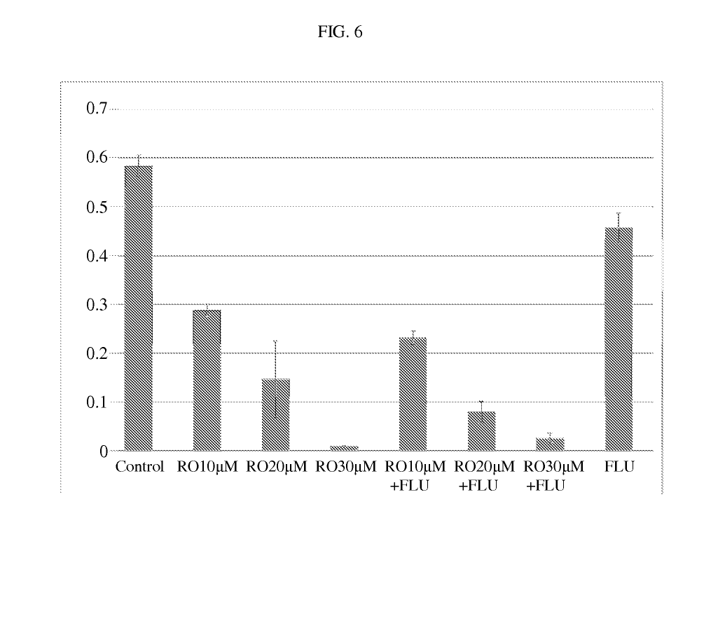

5. Inhibition on cell viability after treatments with Ro-48-8071 alone and in combination with Fludarabine. Refer to FIG. 6, which shows the cell viability data with MTT assay after treatments with Ro-48-8071 (another inhibitor of OSC) and in combination with Fludarabine. As shown in FIG. 6, the treatment with Ro-48-8071 alone inhibits Mec-2 cell proliferation significantly, and a dose dependent sensitivity is also observed with 30 μM of Ro-48-8071 giving the highest inhibition. The combination treatments are shown to be more effective than the Fludarabine treatment alone.

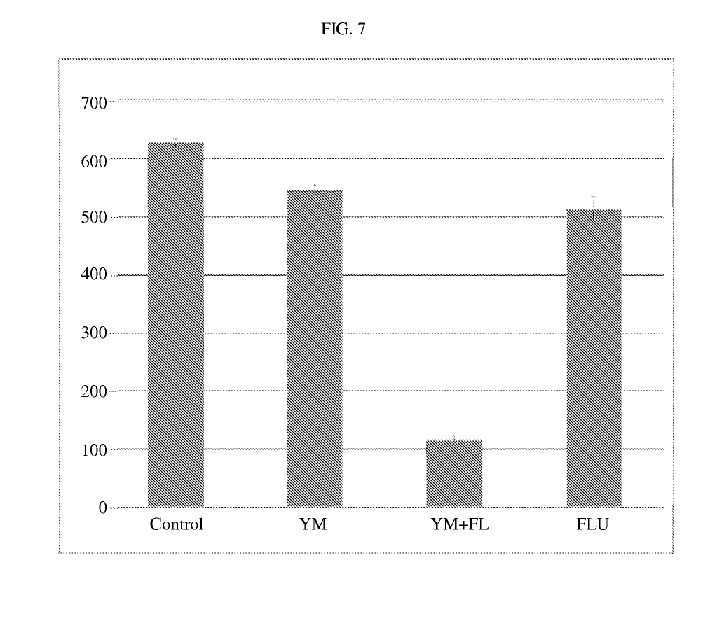

6. Inhibition of cell viability after treatments with YM-53601 alone and in combination with Fludarabine. Refer to FIG. 7, which shows the cell viability data after treatments with YM-53601, an inhibitor of squalene synthase, alone and in combination with Fludarabine. As shown in FIG. 7, the treatment with YM-53601 alone reduces Mec-2 cell proliferation moderately, while the combination treatment with Fludarabine inhibits the cell proliferation significantly. Thus, the results again demonstrate that this class of enzyme inhibitors may act as sensitizers to eliminate or reduce drug-resistance of the cancer cells.

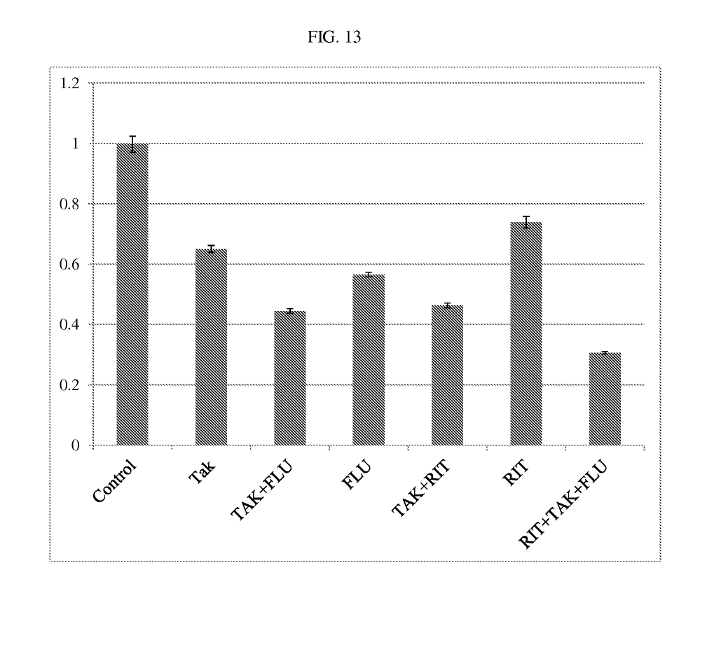

7. Inhibition of cell viability after treatments with TAK-475 alone and in combination with Fludarabine and Rituximab. Refer to FIG. 13, which shows the cell viability data after treatments with 10 μM TAK-475, an inhibitor of squalene synthase, alone and in combination with 10 μM Fludarabine and 10 μg/mL Rituximab. As shown in FIG. 13, the treatment with TAK-475 alone reduces Mec-2 cell proliferation moderately, as does treatment with Fludatibine and, to a lesser extent, Rituxan. However, the combination treatment of TAK-475 with Fludarabine or Rituxan inhibits the cell proliferation more markedly, and the combination of all three drugs even more significantly. This data demonstrates that TAK-475 sensitizes the Mec-2 cells to Rituximab treatment, which may suggest that the TAK-475 inhibits cholesterol biosynthesis, lowers the concentration of membrane cholesterol, and up-regulates CD-20 surface expression, thus making the cells more responsive to the CD-20 inhibitor, Rituximab. The data also demonstrate that less Fludarabine may be needed during treatment. Thus, the results again demonstrate that this class of enzyme inhibitors may act as sensitizers to eliminate or reduce drug-resistance of the cancer cells.

II. Treatments of the inhibitor alone and in combination with existing chemotherapeutic agents on Wac-3 cells.

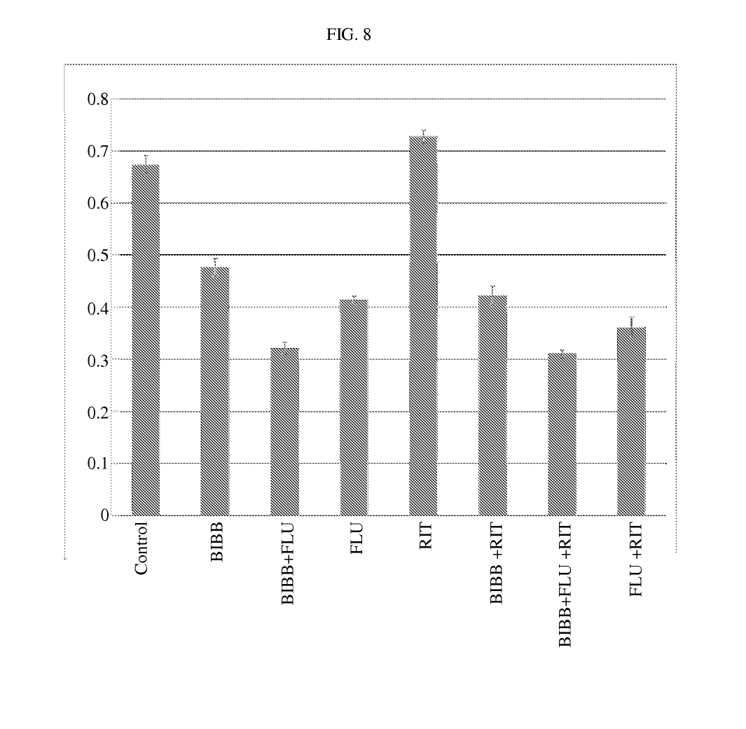

1. Inhibition of cell viability after treatments with BIBB515 alone and in combination with Fludarabine and Rituxan. Refer to FIG. 8, which shows the cell viability data on Wac-3 cells with MTT assay after BIBB515 and various combination treatments with Fludarabine and Rituximab separately and in combination. Wac-3 is another CLL cell line that is response to Fludarabine but resistant to Rituximab. As shown in FIG. 8, the treatment with BIBB515 sensitizes the Wac-3 cells to Rituximab treatment, which may suggest that the BIBB515 inhibits cholesterol biosynthesis, lowers the concentration of membrane cholesterol, up-regulates CD-20 surface expression, thus making the cells more responsive to the CD-20 inhibitor, Rituximab. Also shown in FIG. 8, the combination treatment with BIBB515 and Fludarabine increases inhibition comparing to the Fludarabine treatment alone, thus potentially less Fludarabine is needed during a treatment.

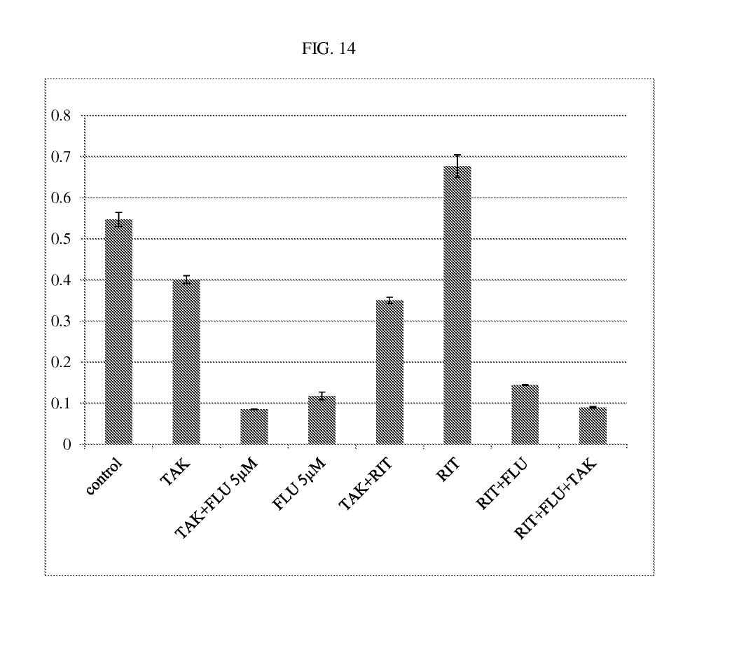

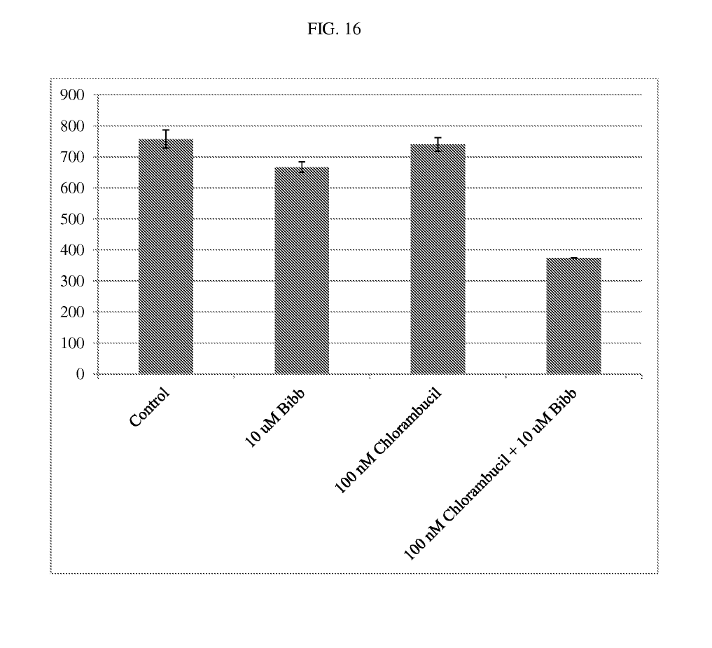

2. Inhibition of cell viability after treatments with TAK-475 alone and in combination with Fludarabine and Rituxan. Refer to FIG. 14, which shows the cell viability data on Wac-3 cells with MTT assay after 10 μM TAK-475 and various combination treatments with 5 μM Fludarabine and 10 μg/mL Rituximab separately and in combination. As shown in FIG. 14, Rituximab treatment alone was wholly ineffective at preventing cellular proliferation, yet the treatment with TAK-475 sensitizes the Wac-3 cells to Rituximab treatment, which may suggest that the TAK-475 inhibits cholesterol biosynthesis, lowers the concentration of membrane cholesterol, up-regulates CD-20 surface expression, thus making the cells more responsive to the CD-20 inhibitor, Rituximab. Also shown in FIG. 16, the combination treatment with TAK-475 and Fludarabine increases inhibition comparing to the Fludarabine treatment alone; thus, less Fludarabine may be needed during treatment.

3. Inhibition of cell viability after treatments with YM-53601 alone and in combination with Fludarabine and Rituxan. Cell viability was also assayed in Wac-3 cells with MTT assay after 10 μM YM-53601 and 10 μg/mL alemtuzumab, separately and in combination. YM-53601 treatment alone was effective at preventing cellular proliferation; whereas the control cell population more than tripled, the YM-53601-treated population did not achieve even a doubling. Treatment with alemtuzumab appeared to be ineffective at the concentration tested in this cell type, and combination treatment yielded a doubling of cell population—apparently no more effective than YM-53601 alone. However, alemtuzumab is known to be efficacious against CD52-bearing cancer cells, such as CLL, CTCL, and TCL, whereas Wac-3 cells doe not bear CD52. Thus, future experiments might demonstrate efficacy of alemtuzumab at a higher concentration and/or in a different cell type.

III. Treatments of the inhibitor alone and in combination with existing chemotherapeutic agents on Burkitt lymphoma cells.

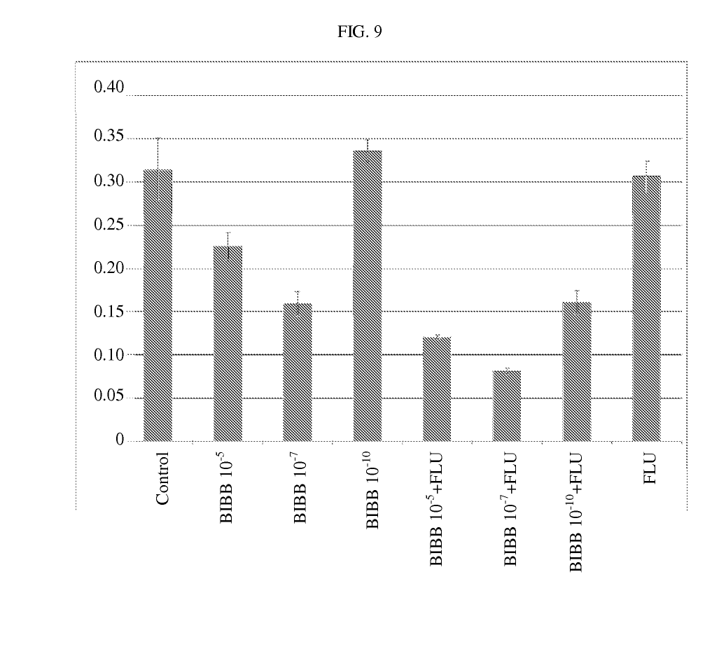

1. Inhibition on cell viability of Raji after treatments with BIBB515 alone and in combination with Fludarabine. Burkitt lymphoma is a highly aggressive lymphoma, and Raji cells are sensitive to Fludarabine treatment at 10 μM concentration. Refer to FIG. 9, which shows Raji cell viabilities after BIBB515 exposure and combination treatments at 72 hours. As shown in FIG. 9, the Raji cell proliferation is reduced by treatment of BIBB515 alone, even at a very low dose (10⁻⁷), and more significantly with the combination treatment. Fludarabine was used at 5 μM concentration.

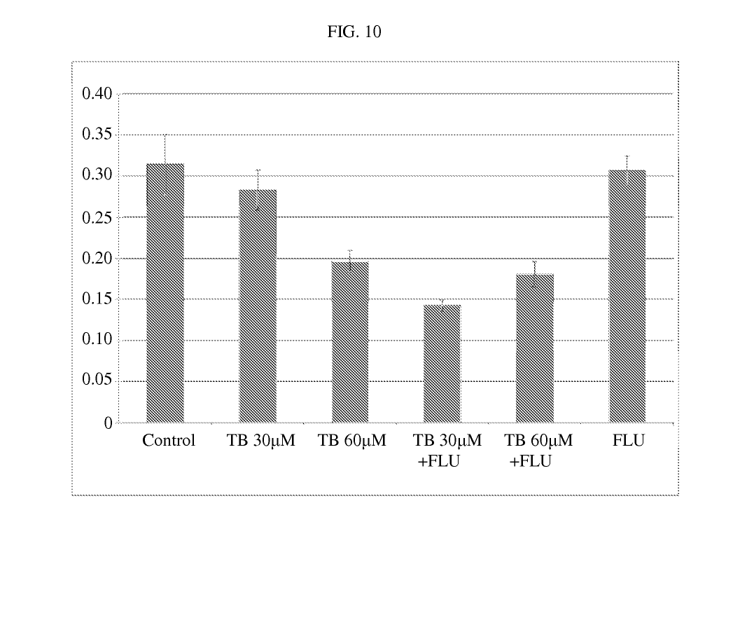

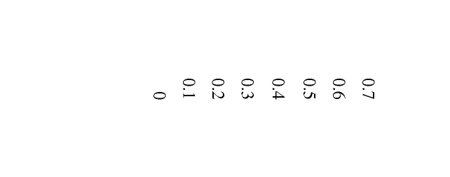

2. Inhibition on cell viability of Raji after treatments with Terbinafine alone and in combination with Fludarabine. Refer to FIG. 10, which shows Raji cell viabilities after Terbinafine and combination treatments in 72 hours. As shown in FIG. 10, the Raji cell proliferation is reduced by treatment with Terbinafine alone and more significantly with the combination treatment. The combination treatment at 30 μM Terbinafine achieves better reduction than the treatment at 60 μM, which may indicate that the reduction of cholesterol concentration may need to be controlled at a certain range to achieve the best sensitizing results. The preferred cholesterol concentration range may also various between different cancer types.

IV. Treatments of the inhibitor alone and in combination with existing chemotherapeutic agents on breast cancer cell line MCF-7.

Among the available chemotherapeutic agents for breast cancer treatments are Tamoxifen and Anastrazole. Tamoxifen a SERM (selective estrogen receptor modulator) is an antagonist of the estrogen receptor in breast tissue via its active metabolite, hydroxytamoxifen. It has been the standard endocrine (anti-estrogen) therapy for hormone receptor-positive early breast cancer in pre-menopausal women. Additionally, it is the most common hormone treatment for male breast cancer. It is also approved by the FDA for the prevention of breast cancer in women at high risk of developing the disease. It has been further approved for the reduction of contralateral (in the opposite breast) cancer. Aromatase inhibitors, for example Anastrazole, have been recently developed. This class of drug is used in the treatment of breast cancer and ovarian cancer in postmenopausal women. MCF-7 cells are breast cancer cells and resistant to treatment of Tamoxifen and Anastrazole at low doses.

A substantial drawback to the efficacy of Tamoxifen and other SERMs, and Anastrazole and other aromatase inhibitors, is that patients develop resistance to these drugs. However, the treatment of BIBB515 alone or in combination with Tamoxifen or Anastrazole induces cell death in MCF-7 cells at relatively low doses of Tamoxifen or Anastrazole.

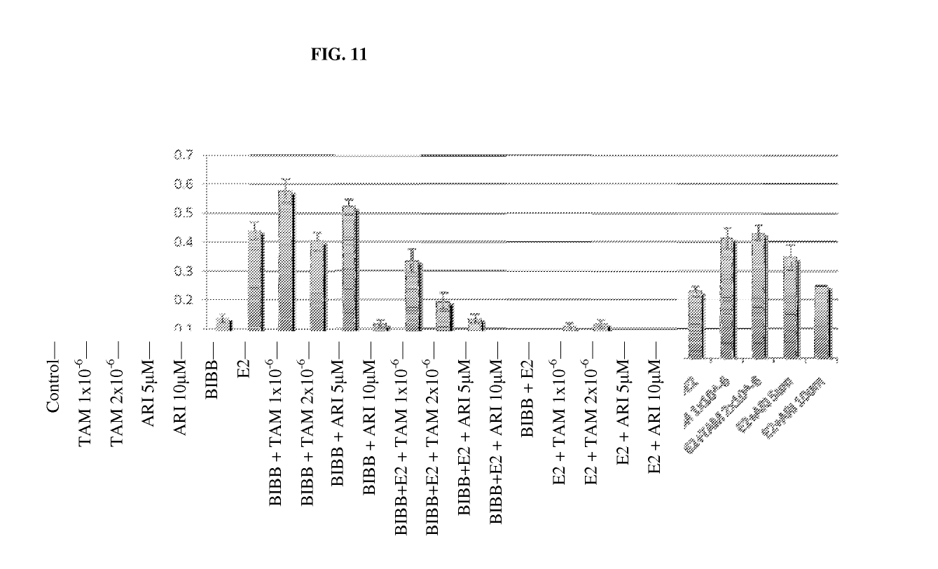

Refer to FIG. 11, which compares MCF-7 cell proliferation data among the treatments of BIBB515, Tamoxifen, Anastraozole, and various combinations thereof. During the experiments, the MCF-7 cells were treated with charcoal-stripped serum prior to treatment and grown with treatment repeated every 72 hrs. The cells were grown for 6 days, on day 7 the cell proliferation was determined by adding MTT and read at 570 nM according to the manufactures instructions. As shown in FIG. 11, MCF-7 cells are sensitized by BIBB515, and the proliferation of cells with estrogen is blocked by BIBB515. Thus, inhibiting the cholesterol pathway can inhibit the estrogen induced cell proliferation in MCF-7 cells.

Combining Anastrazole (or other aromatase inhibitors) and/or Tamoxifen (or other SERMs) with BIBB515/YM/TAK-475 as a new drug combination for metastatic/resistant breast cancer. As seen from the results of combination of BIBB515 sensitizes resistant breast cancer cells to chemotherapy/hormonal therapy. Anastrazole (aramidex) and tamoxifen resistance can be overcome when combined with inhibitors of cholesterol biosynthesis. Similarly, Tak-475 can be used in humans and so its combination with anastrazole and tamoxifen can be developed into treatment regimens for breast cancer. Tamoxifen is typically used at 20 mg/day, and Aramidex at 1 mg/day. TAK-475 has been used at 100 mg/day in Phase II clinical trials. However, it is expected that the use of these three agents in combination may permit lower doses of each to be administered as well, while maintaining efficacy and preventing the development of drug resistance.

V. Treatments of the inhibitor alone and in combination with existing chemotherapeutic agents on multiple myeloma (MM) cancer cell line.

Multiple myeloma (MM) is a clonal B-lymphocyte malignancy, which is characterized by the accumulation of terminally differentiated antibody-producing cells in the bone marrow. Because current treatments offer only a median survival of 3 years, investigators continue to search for novel therapeutic strategies to combat the disease.

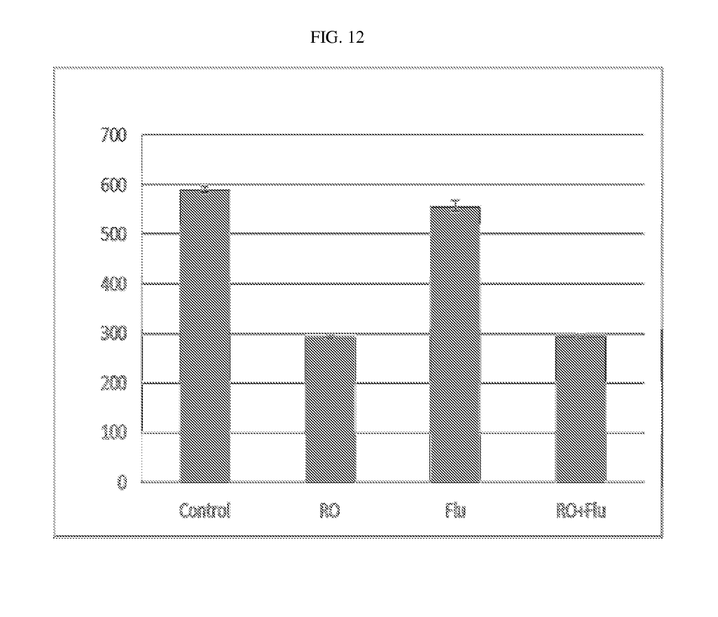

Refer to FIG. 12, which shows the cell proliferation data of MM cells after treatments with Ro-48-8071 and in combination with Fludarabine. As shown in FIG. 12, the treatment with Ro-48-8071 alone provides reduction on cell proliferation, which may suggest that Ro-48-8071 may be able to maintain a static disease and may extend survival.

VI. Treatments of the inhibitor alone and in combination with existing chemotherapeutic agents on patient CLL cells.

1. Inhibition effect with BIBB515 alone and in combination with Fludarabine and Rituximab on patient cells. To assess the effect of treatment on cell viabilities, patient CLL cells were treated with BIBB515 alone and in combination with Fludarabine and Rituximab. Untreated CLL patient cell samples and samples of patients exposed to Fludarabine were used. The treatment was done 24 hrs after the cells plated in T25 flasks. BIBB515 alone was capable of reducing the cell viability moderately, while the inhibition of the cholesterol pathway with BIBB515 sensitizes the cells to Rituximab and Fludarabine from a viability of 70% in the control to 48% in the BIBB515/Rituximab treatment arm and 52% in BIBB515/Fludarabine treatment arm. The three-way combination, which does not result in significant additional reduction, may due to the fact that this patient was previously treated with Fludarabine and might not be responsive to it anymore.

2. Inhibition effect of Ro-48-8071 treatment alone and in combination with Fludarabine and Rituximab on patient cells. To assess the effect of treatment on cell viabilities, patient CLL cells were treated with Ro-48-8071 alone and in combination with Fludarabine and Rituximab. Ro-48-8071 alone was capable of reducing the cell viability from over 80% in the control to about 65%. Additionally, the inhibition of the cholesterol pathway with Ro-48-8071 sensitized the cells to Rituximab and Fludarabine from a viability of over 80% in the control to about 60% in the Ro-48-8071/Rituximab treatment arm and about 65%% in Ro-48-8071/Fludarabine treatment arm. The three-way combination yielded the best results, with a reduction in viability from over 80% in the control to about 55%. This further demonstrates that inhibition of cholesterol pathway alone or in combination with other available chemotherapeutic treat significantly reduces the cancer cell growth and viability.

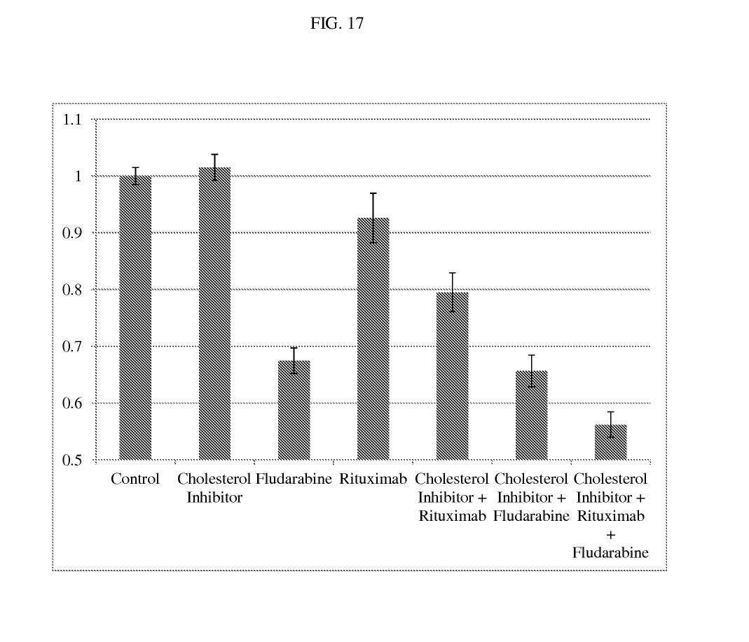

3. Inhibition effect of cholesterol inhibitors (combined) treatment alone and in combination with Fludarabine and Rituximab on patient cells. Refer to FIG. 17, which shows the cell viability of patient's CLL cells treated with a cholesterol inhibitor (BIBB515, YM, TAK-475 and Ro-48-8071 combined data from 6 patients) alone and in combination with Fludarabine and Rituximab. As shown in FIG. 17, the cell proliferation data further demonstrates that inhibition of cholesterol pathway alone or in combination with other available chemotherapeutic treat significantly reduces the cancer cell growth and viability.

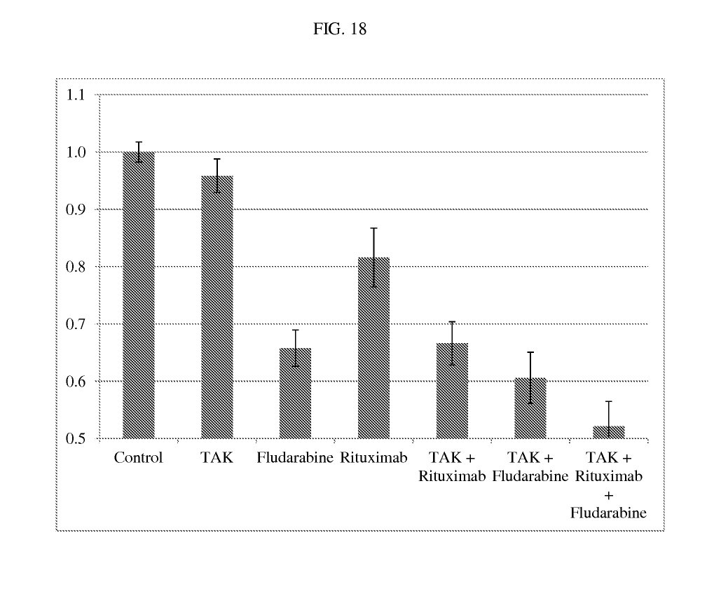

4. Inhibition effect of TAK-475 treatment alone and in combination with Fludarabine and Rituximab on patient cells. Refer to FIG. 18, which shows the cell viability of patient's CLL cells treated with TAK-475 (combined data from 4 patients) alone and in combination with Fludarabine and Rituximab. As shown in FIG. 18, the cell proliferation data further demonstrates that inhibition of cholesterol pathway alone or in combination with other available chemotherapeutic treat significantly reduces the cancer cell growth and viability.

VII. Confocal microscopic analysis of the effect of cholesterol biosynthetic pathway inhibitors on CD20 expression and glycosphingolipid density in Mec-2 cells.

Mec-2 cells were treated with BIBB515, YM-53601, or 10 μM of TAK-475 and imaged by confocal miscrscope. Analysis by confocal microscopy demonstrated an increase in of glycosphingolipids and thus lipid raft density relative to control; this increase was confirmed by flow cytometry in Mec-2 cells treated with YM-53601. (Data not shown.) One way in which this data is significant is that once activated by rituximab, CD20 is redistributed to raft microdomains and that interaction between CD20 and raft membrane protein components results in the activation of the transmembrane signaling machinery. This would explain why rituximab is of limited or no efficacy in Mec-2 and Wac-3 cells, and provides a rationale for the efficacy of combination treatment with rituximab and a cholesterol biosynthetic pathway inhibitor. Additionally, the results suggest the feasibility of inhibiting cancer cell growth with TAK-475 alone and in combination with agents that target CD-20 and CD-52.

VIII. Treatment with inhibitors targeting the cholesterol biosynthetic pathway alone and in combination with existing chemotherapeutic agents on dog lymphoma cells.

Spontaneously occurring lymphoma in the dog has many of the same histopathological, molecular, and clinical features as non-Hodgkin's lymphoma (NHL) in humans. Most of the lymphoma subtypes recognized in humans have histopathologically identical counterparts in the dog and recent investigations show similar molecular characteristics in the two species. Likewise, spontaneous lymphoma in the dog has a similar clinical presentation, response to chemotherapy, and clinical progression compared to NHL in human patients. Given these similarities and the advantage of larger subject size and presence of spontaneous disease (in contrast to small subject size and experimentally induced disease in rodents), spontaneously occurring lymphoma in the dog represents an excellent large animal model for the study of lymphoma in people, including investigation of new therapeutic agents. A canine lymphoma cell line, OSW, was established from the thoracic effusion of a dog with relapsed peripheral T-cell lymphoma. Thus, the cells used to establish this cell line have several of the features associated with enhanced success for establishing human leukemia/lymphoma cell lines. See, e.g., U.S. Pat. No. 7,897,150 and Kisseberth, W. C., et al., “A novel canine lymphoma cell line: a translational and comparative model for lymphoma research,” Leuk Res, 2007 31(12): p. 1709-20.

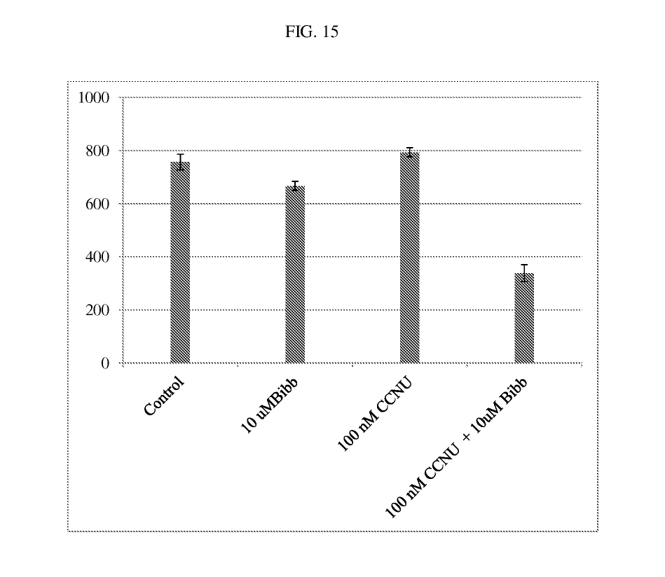

1. Inhibition effect with BIBB515 alone and in combination with lomustine on dog lymphoma cells. Refer to FIG. 15, which shows the cell viabilities of canine lymphoma cells treated with BIBB515 alone and in combination with lomustine. As shown in FIG. 15, BIBB515 alone is capable of reducing the cell viability somewhat, while the inhibition of the cholesterol pathway with BIBB515 sensitizes the cells to lomustine markedly in the BIBB515/Lomustine treatment arm, resulting in synergistic toxicity.

2. Inhibition effect with BIBB515 alone and in combination with Chlorambucil on dog lymphoma cells. Refer to FIG. 16, which shows the cell viabilities of canine lymphoma cells treated with BIBB515 alone and in combination with chlorambucil. As shown in FIG. 16, BIBB515 alone is capable of reducing the cell viability somewhat, while the inhibition of the cholesterol pathway with BIBB515 sensitizes the cells to chlorambucil markedly in the BIBB515/chlorambucil treatment arm, resulting in synergistic toxicity.

Together, the studies in canine lymphoma cells indicates that cholesterol biosynthesis inhibitors are effective in combination with standards of care in the treatment of lymphomas and leukemias. These combinations may be exceptionally effective in cancers which have developed chemotherapeutic resistance.

IX. Treatment with inhibitors targeting the cholesterol biosynthetic pathway alone and in combination with existing chemotherapeutic agents alter membrane lipid content.

Lipids were extracted by the following chloroform and methanol method. To 0.8 parts cells in aqueous solution, 1 part chloroform and 2 parts methanol was added and shaken well, then another 1 part chloroform and 1 part water added and shaken. The mixture was then centrifuged at low speeds for 5-10 min. and the lower layer removed. One part chloroform was added 3 times, shaken and centrifuged to again remove the lower layer. The layers were then combined, washed with KCl and then water. The extracted lipids from Mec-2 cells were vacuum dried after 24 hrs. of treatment with BIBB(10⁻⁵ M) and YM(10⁻⁵ M). Controls were treated with DMSO.

Samples were analyzed by mass spectrometry by the Kansas Lipidomics Research Center (KLRC) and identified and quantified by comparison against standard molecular weights of lipids. Each sample was loaded, vaporized, and ionized by electron beam; ions electromagnetically separated according to mass-to-charge ratio (m/z) were detected, and the ion signal processed into mass spectra.

Compounds were identified based on m/z of the intact ion and the mass of one fragment formed in the mass spectrometer: typically, for polar lipids, a head group fragment; for sphingolipids, a fragment characteristic of the long-chain base or sugar(s); for neutral lipids and for specialized analyses, often an acyl fragment. Quantities were reported as normalized signal/(tissue metric nanomole/3 million cells) against internal standards added in known amounts, usually with an adjustment for variation in response with m/z. Thus, quantities reported as normalized signal/(tissue metric) for diacyl or monoacyl phospholipids can be considered to be equal to nmol/(tis sue metric).

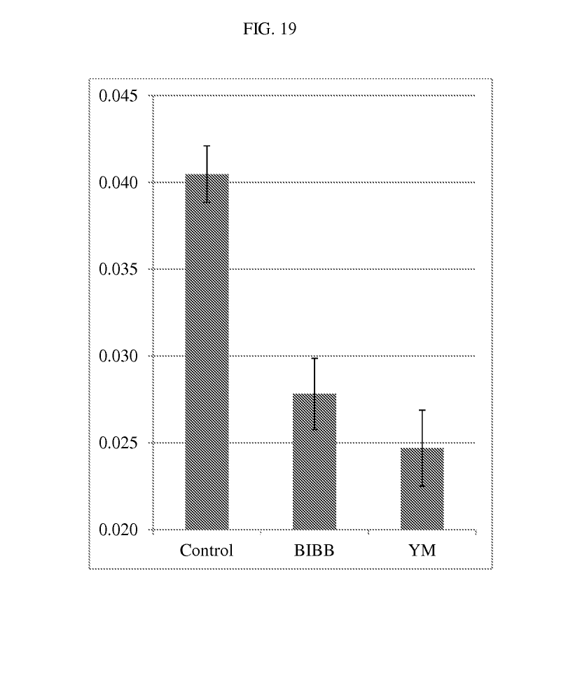

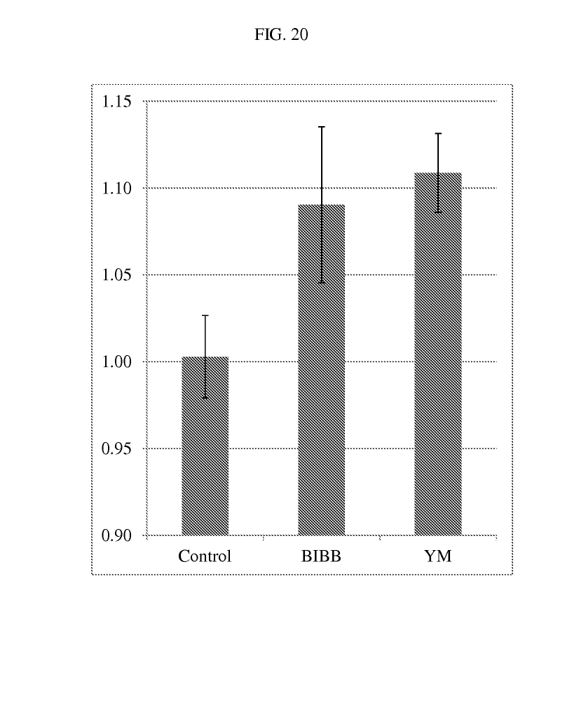

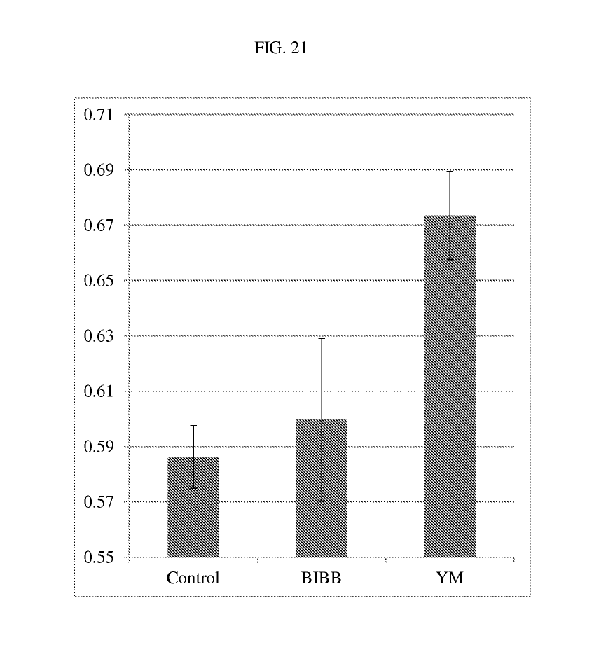

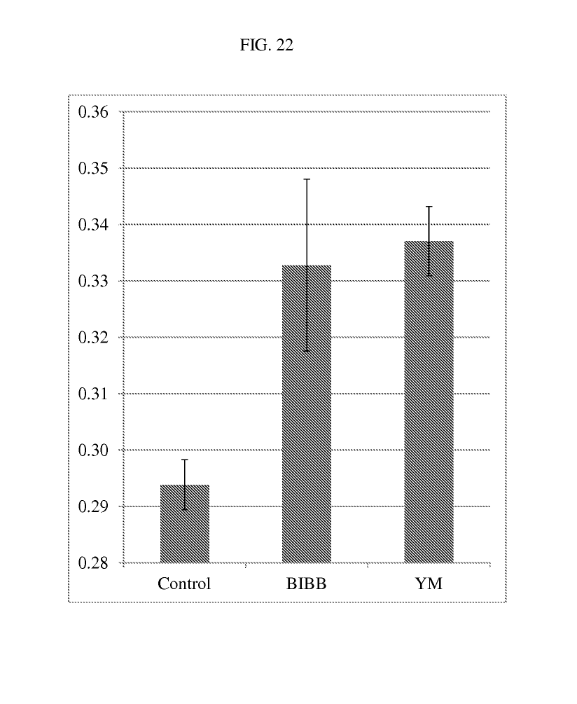

Treatments with BIBB and YM appear to reduce lysophosphatidylcholines (LPC) and some diacyl phosphatidylcholine species, and increase levels of phosphatidylcholines (PC), glycosphingolipids/dihydroglycosphingolipids, and ether-linked phosphatidylcholines; See FIGS. 19-22, respectively. Treatments are also expected to increase some alk(en)yl-acyl PC species. LPC is important because although it is a minor phospholipid in cell membranes (<3%) and in blood plasma (8-12%), it can change surface properties of cells. Analogues of LPC, such as edelfosine, milefosine and perifosine, are also expected to be useful in combinations disclosed herein.

All references, patents or applications, U.S. or foreign, cited in the application are hereby incorporated by reference as if written herein in their entireties. Where any inconsistencies arise, material literally disclosed herein controls.

While the invention has been described in connection with specific embodiments thereof, it will be understood that the inventive methodology is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth and as follows in scope of the appended claims.

What is claimed is:

1-91. (canceled)

92. A method of treatment of hematologic cancer comprising the administration of a therapeutically effective amount of a cholesterol biosynthetic pathway inhibitor.

93. The method of claim 92, wherein the cancer is treatment-resistant.

94. The method of claim 92, wherein the inhibitor inhibits an enzyme regulating the cholesterol biosynthetic pathway.

95. The method of claim 92, wherein the inhibitor is selected from the group consisting of an oxidosqualene cyclase inhibitor, a squalene epoxidase inhibitor, a squalene synthase inhibitor, and combinations thereof.

96. The method of claim 95, wherein the inhibitor is selected from the group consisting of BIBB515, Ro-48-8071, Terbinafine, YM-53601, TAK-475, and any derivative or combinations thereof.

97. The method of claim 92, wherein the cancer is a hematologic cancer selected from the group consisting of leukemia, lymphoma, and myeloma.

98. A method of reducing cancer cell proliferation or viability comprising the step of altering membrane lipid composition of a cancer cell, wherein either:
the cancer cell is a hematologic cancer cell; or
the altering step is carried out by the administration of an inhibitor of squalene epoxidase or an inhibitor of squalene synthase.

99. The method of claim 98, further comprising the step of treating the cancer cell with an existing chemotherapeutic agent or therapeutic monoclonal antibody, sequentially or simultaneously with the altering step.

100. The method of claim 98, wherein the inhibitor is selected from the group consisting of BIBB515, Ro-48-8071, Terbinafine, YM-53601, TAK-475, and any derivative or combinations thereof.

101. The method of claim 98, wherein the cancer cell is from a hematologic cancer selected from the group consisting of leukemia, lymphoma, and myeloma.

102. The method of claim 101, wherein the hematologic cancer is treatment-resistant.

103. A method of treating cancer comprising the step of administering:
an inhibitor of an enzyme regulating membrane cholesterol or membrane lipid composition; and
an additional, chemotherapeutic agent, either together or sequentially.

104. The method of claim 103, wherein the additional, chemotherapeutic agent is selected from the group consisting of an agent for the treatment of hematologic malignancies, an inhibitor of DNA synthesis, a purine analog, fludarabine, a CD20-targeting monoclonal antibody, ocrelizumab, ofatumumab, rituximab, an alkylating antineoplastic agent, chlorambucil, an aromatase inhibitor, anastrozole, an estrogen receptor (ER) antagonist or selective ER modulator (SERM), tamoxifen, or any combination thereof.

105. A pharmaceutical composition comprising
an inhibitor of an enzyme regulating membrane cholesterol or membrane lipid composition; and
an additional, chemotherapeutic agent, together with a pharmaceutically acceptable carrier.

106. The pharmaceutical composition of claim 105, wherein the additional, chemotherapeutic agent is an agent for the treatment of a solid tumor or a hematologic cancer.

107. The pharmaceutical composition of claim 105, wherein the additional, chemotherapeutic agent is selected from the group consisting of an agent for the treatment of hematologic malignancies, an inhibitor of DNA synthesis, a purine analog, fludarabine, a CD20-targeting monoclonal antibody, ocrelizumab, ofatumumab, rituximab, an alkylating antineoplastic agent, chlorambucil, an aromatase inhibitor, anastrozole, an estrogen receptor (ER) antagonist or selective ER modulator (SERM), tamoxifen, or any combination thereof.

108. The pharmaceutical composition of claim 105, wherein the inhibitor inhibits an enzyme regulating the cholesterol biosynthetic pathway.

109. The pharmaceutical composition of claim 105, wherein the inhibitor is selected from the group consisting of an oxidosqualene cyclase inhibitor, a squalene epoxidase inhibitor, a squalene synthase inhibitor, and combinations thereof.

110. The pharmaceutical composition of claim 106, wherein the hematolgic cancer is selected from the group consisting of leukemia, lymphoma, and myeloma.

111. The pharmaceutical composition of claim 106, wherein the solid tumor is breast cancer.
